(19) United States
(12) Patent Application Publication
(10) Pub. No.: US 2012/0164357 A1
(43) Pub. Date: Jun. 28, 2012

(54) External Preparation

(75) Inventors: Daisuke Suzuki, Yokohama-shi (JP); Junko Tamada, Yokohama-shi (JP); Nobuyoshi Koga, Yokohama-shi (JP)

(73) Assignee: Shiseido Company, Ltd., Tokyo (JP)

(21) Appl. No.: 13/393,702

(22) PCT Filed: Jul. 23, 2010
(86) PCT No.: PCT/JP2010/062438

§ 371 Date: Mar. 1, 2012

(30) Foreign Application Priority Data
Sep. 17, 2009 (JP) 2009-215660
Jul. 14, 2010 (JP) 2010-159364

(51) Int. Cl. B32B 1/08; A61Q 19/00; A61K 8/92

(52) U.S. Cl. 428/34.1; 514/561

(57) ABSTRACT

The present invention is an external preparation that comprises (A) a higher alcohol, (B) 0.01-5 wt % of a long chain acyl sulfonate anionic surfactant represented by the following general formula (1), such as stearoyl methyltaurate, and (C) 0.1-5 wt % of tranexamic acid, and has a pH of 3.0-6.0.

R¹CO-a-(CH₂)nSO₃M¹ (1)

[R′CO— denotes a saturated or unsaturated fatty acid residue (acyl group) having 10-22 carbon atoms on average; a denotes —O— or —NR²— (R² denotes a hydrogen atom or an alkyl group having 1-3 carbon atoms); M¹ denotes a hydrogen atom, alkali metal, alkali earth metal, ammonium, or organic amine; n denotes an integer 1-3.]

The object of the present invention is to provide an external preparation containing tranexamic acid that does not cause precipitation of tranexamic acid crystals.

### TECHNICAL FIELD

The present invention relates to an external preparation and more specifically relates to an external preparation comprising tranexamic acid wherein crystalline precipitation of tranexamic acid is prevented.

### BACKGROUND ART

Tranexamic acid and salts thereof are known to be effective against rough skin and skin that chaps easily as well as having an anti-pigment precipitation effect (refer to Patent Document 1); they are also widely used as a whitening agent.

However, tranexamic acid has a high crystallinity and a tranexamic acid-containing emulsion with a dispenser style container had a shortcoming in that the product adhered to the mouth of the container would dry up and hard tranexamic acid crystals would cause clogging or a granular sensation due to the crystals mixed into the dispensed emulsion. This crystal precipitation is not so conspicuous when the blend ratio of tranexamic acid is about 1 wt %, but adhesion of the precipitated crystals to the bottle mouth becomes conspicuous for products having 2 wt %. Therefore, conventionally the container shape is modified to prevent the product from drying up as time passes; however, it would be convenient if research of the base agent in the content leads to development of a base agent in which crystals do not easily precipitate even when a relatively large amount of tranexamic acid is blended in.

An example of prior art technology similar to the base agent configuration of the present invention is Patent Document 2 (refer to paragraph [0037]), which describes a formulation containing tranexamic acid and sodium stearoyl methyltaurate; however, in this system the pH is not controlled so the precipitation of crystals cannot be avoided.

### PRIOR ART DOCUMENTS

### {Patent Documents}

Patent Document 1: JP H1-93519 A

Patent Document 2: JP 2007-246442 A

### SUMMARY OF INVENTION

### Problem that Invention is to Solve

The object of the present invention is to solve the aforementioned problem of conventional tranexamic acid-containing external preparations and provide an external preparation that does not cause precipitation of tranexamic acid crystals.

### Technical Solution

The inventors conducted earnest research to solve the aforementioned problem and discovered that an external preparation containing a specific amount of sodium stearoyl methyltaurate and a higher alcohol whose pH is adjusted to 3-6 can prevent the crystal precipitation of tranexamic acid and thus completed the present invention.

The present invention is an external preparation that characteristically comprises the following (A)-(C) and has a pH of 3-6:
(A) A Higher alcohol
(B) Long chain acyl sulfonate anionic surfactant represented by the following general formula (1) 0.01-5 wt %

R¹CO-a-(CH₂)nSO₃M¹ (1)

[In formula (1), R′CO— denotes a saturated or unsaturated fatty acid residue (acyl group) having 10-22 carbon atoms on average; a denotes —O— or —NR²— (wherein R² denotes a hydrogen atom or an alkyl group having 1-3 carbon atoms); M¹ denotes a hydrogen atom, alkali metal, alkali earth metal, ammonium, or organic amine; n denotes an integer 1-3.]
(C) Tranexamic acid 0.1-5 wt %

Also, the present invention is a product for external use wherein the aforementioned external preparation is contained in a container having a dispenser.

### ADVANTAGEOUS EFFECTS

The external preparation of the present invention does not cause precipitation of crystals even when the blend ratio of tranexamic acid is relatively high.

Also, the product for external use of the present invention has superior usability because it does not cause clogging or granular sensation due to crystals mixed into the dispensed liquid and also is equipped with a dispenser.

### MODE FOR CARRYING OUT THE INVENTION

The embodiments of the present invention are described below.

### ((A) Higher Alcohol)

Selection of (A) a higher alcohol used in the present invention is not limited in particular as long as it can be used in the field of cosmetics, medical drugs, quasi-drugs, etc.; examples include saturated straight chain monovalent alcohols and unsaturated monovalent alcohols. Examples of the saturated straight chain monovalent alcohol include dodecanol (=lauryl alcohol), tridecanol, tetradecanol (=myristyl alcohol), pentadecanol, hexadecanol (=cetyl alcohol), heptadecanol, octadecanol (=stearyl alcohol), nonadecanol, icosanol (=arachyl alcohol), henicosanol, docosanol (=behenyl alcohol), tricosanol, tetracosanol (=carnaubyl alcohol), pentacosanol, and hexacosanol (=ceryl alcohol). Examples of the unsaturated monovalent alcohol include elaidyl alcohol. In the present invention, a saturated straight chain monovalent alcohol is preferable in terms of stability and such.

For ingredient (A), one, two or more types can be used; it is preferable that the alkyl chain length obtained with arithmetic averaging is 18 or longer. If the average chain length is less than 18, then the melting point of the gel-like composition and the cream composition using it as the outer phase becomes lower and the high temperature stability is sometimes not sufficient. The upper limit of the average alkyl chain length is not limited in particular; a preferable alkyl chain length is about 22.

The blend ratio of ingredient (A) is preferably in a specific ratio with ingredient (B), as discussed later.

### ((B) A Long Chain Acyl Sulfonate Anionic Surfactant)

(B) A long chain acyl sulfonate anionic surfactant used in the present invention is represented by the following general formula (1).

R¹CO-a-(CH₂)nSO₃M¹ (1)

[In formula (1), R¹CO— denotes a saturated or unsaturated fatty acid residue (acyl group) having 10-22 carbon atoms on average; a denotes —O— or —NR²— (wherein R² denotes a hydrogen atom or an alkyl group having 1-3 carbon atoms); M¹ denotes a hydrogen atom, alkali metal, alkali earth metal, ammonium, or organic amine; n denotes an integer 1-3.]

Here, examples of R¹CO— include C₁₁H₂₃CO, C₁₂H₂₅CO, C₁₃H₂₇CO, C₁₄H₂₉CO, C₁₅H₃₁CO, C₁₆H₃₃CO, C₁₇H₃₅CO, coco palm-fatty acid residues, and palm-fatty acid residues. For R¹CO—, those having an average of 12-22 carbon atoms are more preferable from the point of view of safety and such.

“a” denotes —O— or —NR²— (where R² denotes a hydrogen atom or an alkyl group having 1-3 carbon atoms). These are electron donor groups. For a, —O—, —NH—, and —N(CH₃)— are preferable.

M¹ represents a hydrogen atom, alkali metal, alkali earth metal, ammonium, or organic amine or a derivative thereof. Examples of M¹ include lithium, potassium, sodium, calcium, magnesium, ammonium, monoethanolamine, diethanolamine, triethanolamine, sodium taurate, and sodium N-methyltaurate.

n denotes an integer 1-3.

Examples of ingredient (B) that are compounds for which a in the aforementioned general formula (1) denotes —O—, i.e. long chain acyl isethionate anionic surfactants, include cocoyl isethionate, stearoyl isethionate, lauryl isethionate, and myristoyl isethionate.

Examples of compounds for which a in the aforementioned general formula (1) denotes —NH—, i.e. long chain acyl taurate anionic surfactants, include N-lauroyl taurate, N-cocoyl-N-ethanol taurate, N-myristoyl taurate, and N-stearoyl taurate.

Examples of compounds for which a in the aforementioned general formula (1) denotes —N(CH₃)—, i.e. long chain acyl methyltaurate anionic surfactants, include N-lauroyl-N-methyltaurate, N-palmitoyl-N-methyltaurate, N-stearoyl-N-methyltaurate, and N-cocoyl-N-methyltaurate.

Of these, N-stearoyl-N-methyltaurate is particularly preferable for ingredient (B). One, two, or more types of ingredient (B) can be used.

The blend ratio of (B) a long chain acyl sulfonate anionic surfactant is preferably 0.01-5 wt %, more preferably 0.1-3 wt %, relative to the total amount of the external preparation of the present invention. If the blend ratio of ingredient (B) is less than 0.01 wt %, then the effect of maintaining the viscosity of the system is not sufficient; if the blend ratio is more than 5 wt %, then the viscosity may be too high to use a dispenser.

The blend ratio of ingredient (B) is, relative to the tranexamic acid, 0.5 wt % or more, preferably 5 wt % or more and 50 wt % or less.

In the present invention, a stable gel is formed from the aforementioned ingredient (A) and ingredient (B). Because of this, the viscosity is stably maintained even in a low pH region.

The blend ratio between ingredient (A) and ingredient (B) in the present invention is preferably, as a molar ratio between ingredient (A) and ingredient (B), Ingredient (A):
Ingredient (B)=4:1-10:1.

### ((C) Tranexamic Acid)

(C) tranexamic acid used in the present invention has a chemical name of trans-4-aminomethyl-cyclohexanecarboxylic acid and is an ingredient blended into skin cosmetics as an effective ingredient against rough skin and skin that chaps easily as well as pigmentation.

The blend ratio of (C) is 0.1-5 wt % relative to the total amount of the external preparation of the present invention. When the amount of tranexamic acid is small, there is no problem in terms of crystal precipitation and therefore there is no need for the configuration of the present invention. When the blend ratio of tranexamic acid is too high, it becomes impossible to prevent crystal precipitation of the tranexamic acid.

The blend ratio of tranexamic acid is preferably 1-3 wt % and more preferably 1-2 wt %.

In the present invention, it is necessary to maintain the pH of the external preparation in the range of 3.0-6.0. When the pH is lower than 3.0, then the viscosity becomes too low; when the pH is higher than 6.0, (C) tranexamic acid's crystals are more likely to precipitate.

For pH adjustment, organic acids and/or inorganic acids used for common pH adjustment are used. Examples of such acids include citric acid, hydrochloric acid, and lactic acid.

The blend ratio of the acid is such that the pH of the external preparation is maintained at 3.0-6.0.

In the present invention, it is preferable to further blend in (D) a carboxyvinyl polymer. The addition of (D) a carboxyvinyl polymer further increases the effect of suppressing crystal precipitation. When the long chain acyl sulfonate anionic surfactant, which is ingredient (b), is not blended in, neutralizing the carboxyvinyl polymer with a usual neutralizer such as potassium hydroxide or 2-amino-2-methyl-1,3-propanediol (AMPD) would actually promote crystallization of the tranexamic acid when tranexamic acid is added. On the other hand, crystallization is suppressed when the long chain acyl sulfonate anionic surfactant coexists as the neutralizer for the tranexamic acid.

In the present invention, a preferable blend ratio of (D) a carboxyvinyl polymer is 0.01-1 wt %, more preferably 0.05-0.5 wt %.

In the present invention, it is preferable to further blend in (E) a humectant. The addition of (E) a humectant further increases the effect of suppressing crystal precipitation.

Examples of the humectant include dipropylene glycol, propylene glycol, 1,3-butylene glycol, glycerin, polyethylene glycol, xylitol, sorbitol, maltitol, erythritol, POE/POP copolymer dialkyl ether, chondroitin sulfate, hyaluronic acid, mucoitin sulfate, charonic acid, atelocollagen, cholesteryl 12-hydroxystearate, sodium lactate, bile salts, dl-pyrrolidone carboxylates, short chain soluble collagen, diglycerin (E0) P0 adduct, chestnut rose fruit extract, yarrow extract, and sweet clover extract.

In the present invention, a preferable blend ratio of (E) a humectant is 5-20 wt %, more preferably 5-15 wt %.

In the present invention, in addition to the aforementioned ingredients, water and oil components are blended in.

The oil component can be selected from those commonly used for external preparations as long as the stability is not adversely affected. Non-polar oil components such as hydrocarbon oil components or silicone oils are preferable for the oil component. “Non-polar oil component” stands for those which do not have hydration functional groups such as ether bonds, ester bonds, amide bonds, hydroxyl groups, and carboxyl groups.

Examples of the hydrocarbon oil that can be used include liquid paraffin, squalene, squalene, paraffin, isoparaffin, and ceresin.

Examples of the silicone oils include chain-like silicones such as dimethylpolysiloxane, methylphenyl polysiloxane, and methyl hydrogen polysiloxane; ring silicones such as octamethylcyclotetrasiloxane, decamethylcyclopentasiloxane, and dodecamethylcyclohexasiloxane; silicone resins forming a three-dimensional network structure, and silicone rubbers.

Examples of the liquid fats and oils include linseed oil, tsubaki oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, triglycerin, glycerin trioctanoate, and glycerin triisopalmitate.

Examples of the ester oils include cetyl octanoate, hexyl laurate, isopropyl myristate, octyl palmitate, isocetyl stearate, isopropyl isostearate, octyl isopalmitate, isodecyl oleate, glyceryl tri-2-ethylhexanoate, pentaerythritol tetra-2-ethylhexanoate, 2-ethylhexyl succinate, and diethyl sebacate.

The blend ratio of the oil component is not limited in particular; a preferable blend ratio is about 3-25 wt % relative to the total amount of the external preparation.

Various ingredients that are usually used in external preparations can be blended into the external preparation of the present invention as long as the stability is not adversely affected. Examples of such ingredients include, but are not limited to, monovalent alcohols, polyvalent alcohols, water soluble polymers, sequestering agents, antioxidants, perfumes, pigments, and powders.

The external preparation of the present invention can be used for external preparations including skin care cosmetics such as moisture retaining creams, massage creams, cleansing creams, emulsions, and essences, body care cosmetics such as sunscreens and body creams, and gel-like foundations; those having a viscosity of 500-12,000 (mPa·s/30° C.) are preferable and more preferable are those having a viscosity of 1,000-4,000 (mPa·s/30° C.). In terms of the manner of use, the external preparation of the present invention contained in a container having a dispenser is favorable.

### EXAMPLES

The present invention is described in detail below by referring to Examples. The present invention is not limited to these Examples. The blend ratios are in mass-percentage units unless specified otherwise.

Before going into Examples, the evaluation method and the evaluation criteria used in the present invention are described.

### (1) Crystal Precipitation of Tranexamic Acid

The sample was put into a container with a dispenser and let rest at 25° C., 37° C., and 50° C. After this, the content was discharged every week for four weeks and measurements were done to see if there was crystal precipitation. Three samples were used for each temperature and the crystal precipitation rates of the samples were determined as follows. Crystal precipitation was deemed present when crystals were felt as the sample was applied on the hand and deemed not present when crystals were not felt.

Crystal precipitation rate (%)=Total number of samples for which crystal precipitation was present/{(Number of temperature levels×Number of samples at the same temperature×Number of periods)}×100

Here, the number of temperature levels is “3”, i.e. 25° C., 37° C., and 50° C. The number of samples at the same temperature is “3”. The number of periods (weeks) is “4”, i.e. one week, two weeks, three weeks, and four weeks.

### Test Examples 1-11

External preparations were prepared using the formulations shown in the following Tables 1 and 2 and the crystal precipitation rate was measured using the aforementioned method. The results are also shown in Tables 1 and 2.

TABLE 1

| | | Test | Test | Test | Test | Test | Test |
|---|---|---|---|---|---|---|---|
| | | example | example | example | example | example | example |
| Classification | Raw material name | 1 | 2 | 3 | 4 | 5 | 6 |
| Water | Ion-exchanged water | to. 100% | to. 100% | to. 100% | to. 100% | to. 100% | to. 100% |
| Alcohol | Ethyl alcohol | 5.0 | 5.0 | 5.0 | 5.0 | 5.0 | 5.0 |
| Humectant | Glycerin | 5.0 | 5.0 | 5.0 | 5.0 | 5.0 | 5.0 |
| | Dipropylene glycol | 5.0 | 5.0 | 5.0 | 5.0 | 5.0 | 5.0 |
| | 1,3-butylene glycol | 5.0 | 5.0 | 5.0 | 5.0 | 5.0 | 5.0 |
| | Erythritol | 0.1 | 0.1 | 0.1 | 0.1 | 0.1 | 0.1 |
| | Polyoxyethylene (14) | 0.1 | 0.1 | 0.1 | 0.1 | 0.1 | 0.1 |
| | polyoxypropylene (7) | | | | | | |
| | dimethyl ether *1 | | | | | | |
| Thickener | Carboxyvinyl polymer *2 | 0.1 | 0.1 | 0.1 | 0.1 | 0.1 | 0.1 |
| Neutralizer | Potassium hydroxide | 0.05 | 0.05 | 0.05 | 0.05 | 0.05 | 0.05 |
| Activator | Sodium stearoyl | 0.2 | 0.2 | 0.2 | 0.2 | 0.2 | 0.2 |
| | methyltaurate | | | | | | |
| Oil components | Behenyl alcohol | 1.1 | 1.1 | 1.1 | 1.1 | 1.1 | 1.1 |
| | Squalane | 2.0 | 2.0 | 2.0 | 2.0 | 2.0 | 2.0 |
| | Stearyl alcohol | 0.3 | 0.3 | 0.3 | 0.3 | 0.3 | 0.3 |
| | Diglyceryl | 0.6 | 0.6 | 0.6 | 0.6 | 0.6 | 0.6 |
| | diisostearate | | | | | | |
| | Dimethicone *3 | 2.0 | 2.0 | 2.0 | 2.0 | 2.0 | 2.0 |
| | Pentaerythritol tetra- | 1.0 | 1.0 | 1.0 | 1.0 | 1.0 | 1.0 |
| | 2-ethylhexanoate | | | | | | |
| Drugs | Tranexamic acid | 2.0 | 2.0 | 2.0 | 2.0 | 2.0 | 2.0 |
| | L-lysine hydrochloride | 0.01 | 0.01 | 0.01 | 0.01 | 0.01 | 0.01 |
| Chelating agent | EDTA-3Na•2H2O | 0.05 | 0.05 | 0.05 | 0.05 | 0.05 | 0.05 |
| pH adjustment agent | Citric acid | — | 4.0 | 1.0 | 0.7 | 0.4 | 0.1 |
| Preservative | Phenoxy ethanol | 0.5 | 0.5 | 0.5 | 0.5 | 0.5 | 0.5 |
| Stabilizer | Sodium pyrosulfite | 0.003 | 0.003 | 0.003 | 0.003 | 0.003 | 0.003 |
| Characteristic value | Viscosity (mPa · s/30° C.) | 4480 | 1250 | 3100 | 3330 | 2110 | 3050 |
| | pH | 6.9 | 3.4 | 4.7 | 4.9 | 5.3 | 6.0 |

| | | | | | | |
|---|---|---|---|---|---|---|
| Crystal precipitation rate (%) | 54 | 0 | 17 | 25 | 22 | 32 |

TABLE 2

| | | Test | Test | Test | Test | Test | Test |
|---|---|---|---|---|---|---|---|
| | | example | example | example | example | example | example |
| Classification | Raw material name | 1 | 7 | 8 | 9 | 10 | 11 |
| Water | Ion-exchanged water | to. 100% | to. 100% | to. 100% | to. 100% | to. 100% | to. 100% |
| Alcohol | Ethyl alcohol | 5.0 | 5.0 | 5.0 | 5.0 | 5.0 | 5.0 |
| Humectant | Glycerin | 5.0 | 5.0 | 5.0 | 5.0 | 5.0 | 5.0 |
| | Dipropylene glycol | 5.0 | 5.0 | 5.0 | 5.0 | 5.0 | 5.0 |
| | 1,3-butylene glycol | 5.0 | 5.0 | 5.0 | 5.0 | 5.0 | 5.0 |
| | Erythritol | 0.1 | 0.1 | 0.1 | 0.1 | 0.1 | 0.1 |
| | Polyoxyethylene (14) | 0.1 | 0.1 | 0.1 | 0.1 | 0.1 | 0.1 |
| | polyoxypropylene (7) | | | | | | |
| | dimethyl ether *1 | | | | | | |
| Thickener | Carboxyvinyl polymer *2 | 0.1 | 0.1 | 0.1 | 0.1 | 0.1 | 0.1 |
| Neutralizer | Potassium hydroxide | 0.05 | 0.05 | 0.05 | 0.05 | 0.05 | 0.05 |
| Activator | Sodium stearoyl | 0.2 | 0.2 | 0.2 | 0.2 | 3.0 | 0.03 |
| | methyltaurate | | | | | | |
| Oil components | Behenyl alcohol | 1.1 | 1.1 | 1.1 | 1.1 | 1.1 | 1.1 |
| | Squalane | 2.0 | 2.0 | 2.0 | 2.0 | 2.0 | 2.0 |
| | Stearyl alcohol | 0.3 | 0.3 | 0.3 | 0.3 | 0.3 | 0.3 |
| | Diglyceryl | 0.6 | 0.6 | 0.6 | 0.6 | 0.6 | 0.6 |
| | diisostearate | | | | | | |
| | Dimethicone *3 | 2.0 | 2.0 | 2.0 | 2.0 | 2.0 | 2.0 |
| | Pentaerythritol tetra- | 1.0 | 1.0 | 1.0 | 1.0 | 1.0 | 1.0 |
| | 2-ethylhexanoate | | | | | | |
| Drugs | Tranexamic acid | 2.0 | 3.0 | 3.0 | 3.0 | 2.0 | 2.0 |
| | L-lysine hydrochloride | 0.01 | 0.01 | 0.01 | 0.01 | 0.01 | 0.01 |
| Chelating agent | EDTA-3Na•2H2O | 0.05 | 0.05 | 0.05 | 0.05 | 0.05 | 0.05 |
| pH adjustment agent | Citric acid | — | — | — | — | — | — |
| | Lactic acid | — | — | 0.15 | 5.7 | 0.14 | 0.12 |
| Preservative | Phenoxy ethanol | 0.5 | 0.5 | 0.5 | 0.5 | 0.5 | 0.5 |
| Stabilizer | Sodium pyrosulfite | 0.003 | 0.003 | 0.003 | 0.003 | 0.003 | 0.003 |
| Characteristic value | Viscosity (mPa · s/30° C.) | 4480 | 3430 | 3380 | 3210 | 3820 | 1210 |
| | pH | 6.9 | 7.0 | 6.0 | 4.5 | 5.9 | 5.9 |

| | | | | | | |
|---|---|---|---|---|---|---|
| Crystal precipitation rate (%) | 54 | 66 | 33 | 0 | 27 | 22 |

*1: The polyoxyethylene/polyoxypropylene part was randomly polymerized.
*2: Syntalen L (from 3V SIGMA)
*3: Silicone KF-96A-6CS (from Shin-Etsu Chemical Co. Ltd.)

Formulation examples of the external preparation of the present invention are shown below. Needless to say, the present invention is not limited at all by these formulation examples and it is specified by the scope of the claim.

### Formulation Example 1

### Emulsion

| Purified water | Balance | |
|---|---|---|
| Ethyl alcohol | 3 | wt % |
| Glycerin | 5 | |
| Butylene glycol | 5 | |
| Dipropylene glycol | 5 | |
| Carboxyvinyl polymer | 0.1 | |
| Potassium hydroxide | 0.05 | |
| Sodium N-stearoyl-N-methyltaurate | 0.2 | |
| Stearyl alcohol | 0.3 | |
| Behenyl alcohol | 1.1 | |
| Methylpolysiloxane | 2 | |
| Squalane | 2 | |
| Pentaerythritol tetra-2-ethylhexanoate | 1 | |
| Glyceryl diisostearate | 0.6 | |
| Tranexamic acid | 2 | |
| Sodium pyrosulfite | 0.003 | |
| Trisodium edetate | 0.02 | |
| Phenoxy ethanol | 0.5 | |
| Citric acid | 1 | |

### (Preparation Method)

The oil soluble ingredients were dissolved in the oil component and heated up to 70° C. (oil phase). Separately, the water soluble ingredients were dissolved in the purified water and heated up to 70° C. (water phase). The aforementioned oil phase was added and mixed into this water phase, followed by stirring, and cooled down to room temperature. The pH was 4.7.

### Formulation Example 2

| Purified water | Balance |
|---|---|
| Ethyl alcohol | 3 |
| Glycerin | 5 |
| Butylene glycol | 5 |
| Dipropylene glycol | 5 |
| Carboxyvinyl polymer | 1.1 |
| Potassium hydroxide | 0.05 |
| Sodium N-stearoyl-N-methyltaurate | 0.2 |
| Stearyl alcohol | 0.3 |
| Behenyl alcohol | 1.1 |
| Methylpolysiloxane | 2 |
| Squalane | 2 |
| Pentaerythritol tetra-2-ethylhexanoate | 1 |
| Glyceryl diisostearate | 1.6 |
| Tranexamic acid | 2 |
| Sodium pyrosulfite | 0.003 |
| Trisodium edetate | 0.02 |
| Phenoxy ethanol | 0.5 |
| Lactic acid | 0.55 |

### (Preparation Method)

The oil soluble ingredients were dissolved in the oil component and the temperature was raised up to 70° C. (oil phase). Separately, the water soluble ingredients were dissolved in the purified water and heated up to 70° C. (water phase). The aforementioned oil phase was added and mixed into this water phase, followed by stirring, and cooled down to room temperature. The pH was 5.1.

1. An external preparation that characteristically comprises the following (A)-(C) and has a pH of 3.0-6.0:
(A) a higher alcohol;
(B) a long chain acyl sulfonate anionic surfactant represented by the following general formula
(1) 0.01-5 wt % of
R¹CO-a-(CH₂)nSO₃M¹ (1)
in formula (1), R¹CO— denotes a saturated or unsaturated fatty acid residue (acyl group) having 10-22 carbon atoms on average, a denotes —O— or —NR²— (wherein R² denotes a hydrogen atom or an alkyl group having 1-3 carbon atoms), M¹ denotes a hydrogen atom, alkali metal, alkali earth metal, ammonium, or organic amine, n denotes an integer 1-3; and
(C) Tranexamic tranexamic acid 0.1-5 wt %,

2. The external preparation of claim 1, wherein ingredient (B) is a stearoyl methyltaurate.

3. The external preparation of claim 1, which further comprises 0.01-1 wt % of (D) carboxyvinyl polymer.

4. The external preparation of claim 1, which further comprises 5-20 wt % of (E) a humectant.

5. A product for external use wherein the external preparation of claim 1-4 is in a container having a dispenser.

6. The external preparation of claim 2, which further comprises 0.01-1 wt % of (D) carboxyvinyl polymer.

7. The external preparation of claim 2, which further comprises 5-20 wt % of (E) a humectant.

8. The external preparation of claim 3, which further comprises 5-20 wt % of (E) a humectant.

9. A product for external use wherein the external preparation of claim 2 is in a container having a dispenser.

10. A product for external use wherein the external preparation of claim 3 is in a container having a dispenser.

11. A product for external use wherein the external preparation of claim 4 is in a container having a dispenser.
